(19) United States
(12) Patent Application Publication
(10) Pub. No.: US 2015/0065854 A1
(43) Pub. Date: Mar. 5, 2015

(54) JOINT ESTIMATION OF ATTENUATION AND ACTIVITY INFORMATION USING EMISSION DATA

(71) Applicant: General Electric Company, Schenectady, NY (US)

(72) Inventors: Sangtae Ahn, Guilderland, NY (US); Ravindra Mohan Manjeshwar, Glenville, NY (US)

(21) Appl. No.: 13/665,551

(22) Filed: Oct. 31, 2012

(51) Int. Cl. A61B 6/00; G01R 33/385; A61B 6/03; G01R 33/48

(52) U.S. Cl. CPC A61B 6/4417; G01R 33/481; G01R 33/385; A61B 6/037
USPC 600/411

(57) ABSTRACT

Methods, systems and non-transitory computer readable media for imaging are disclosed. Emission projection data corresponding to a target region of a subject is acquired using an emission tomography system. Additionally, one or more magnetic resonance images of the target region are generated using a magnetic resonance imaging system operatively coupled to the emission tomography system. A partially-determined attenuation map is determined by identifying one or more regions in the partially-determined attenuation map with a designated confidence level based on the magnetic resonance images. Further, a complete attenuation map and/or a complete activity map is reconstructed from the emission projection data using the partially-determined attenuation map as a constraint. One or more images corresponding to the target region are then generated based on the partially-determined attenuation map, the complete attenuation map and/or the complete activity map.

### BACKGROUND

Positron emission tomography (PET) finds use in generating images that represent a distribution of positron-emitting nuclides, for example, within a patient's body. During PET imaging, a radionuclide is injected into the patient. As the radionuclide decays, positrons are emitted that collide with electrons, resulting in an annihilation event, which converts the entire mass of the positron-electron pair into two 511 kilo-electron volt (keV) photons emitted in substantially opposite directions along a line of response (LOR). In a PET system, detectors placed along the LOR on a detector ring detect the annihilation photons. Particularly, the detectors detect a coincidence event if the photons arrive and are detected at the detector elements within a coincidence timing window. The PET system uses the detected coincidence information along with other acquired image data for ascertaining localized concentrations of the radionuclide for use in generating a functional diagnostic image.

However, during PET imaging, photon-electron interactions may result in attenuation of emitted photons, which in turn, may lead to degraded image quality and inaccurate PET quantitation. Accordingly, PET imaging is often combined with X-ray computed tomography (CT) imaging to correct for such attenuation. CT produces transmission data of high statistical quality, which often yields an essentially noise-free attenuation map. However, CT imaging may have limited soft-tissue contrast and involve administration of substantial radiation to a patient.

Accordingly, in certain imaging scenarios, non-radiation based imaging, such as magnetic resonance imaging (MRI) may be used in conjunction with PET imaging for generating high-quality images useful for diagnosis and/or treatment. To that end, MRI and PET scans may be performed sequentially in separate scanners or simultaneously in a combined PET/MRI scanner. The unmatched soft tissue contrast that MRI provides along with functional imaging options, such as spectroscopy and functional MRI, complements the molecular information that PET offers with high-sensitivity tracking of biomarkers. Particularly, simultaneous acquisition of PET and MRI data provides unique opportunities to study biochemical processes through fusion of complementary information from the orthogonal MRI and PET imaging modalities.

MRI, however, may not provide a direct transformation of magnetic resonance (MR) images into PET attenuation values. The MR images reflect distribution of hydrogen nuclei with relaxation properties rather than electron density, which is related to PET attenuation. Accordingly, certain conventional approaches employ a segmentation-based approach, where an MR image is segmented into a number of regions and then a predetermined attenuation value is assigned to each segment. Certain other approaches are based on an atlas that includes a template MR image and a corresponding attenuation map, where a patient-specific MR image is registered to the template MR image and the template attenuation map is warped using the same spatial transformation as used for the registration to produce a corresponding patient-specific attenuation map. Other approaches are drawn to joint estimation of PET attenuation and activity maps from PET emission projection data, where all voxels/pixels are initially unknown.

Although bones/metal implants and lungs/air have substantially different PET attenuation values, MRI typically does not discriminate well between bone/metal implants and air/lungs. Use of conventional segmentation-based approaches for estimation of PET attenuation and activity, thus, may result in inaccurate attenuation correction, particularly in and/or near bones and lungs. The atlas-based approaches may require sophisticated data processing techniques such as pattern recognition and machine learning to address significant inter-patient variation in anatomy particularly for patient body parts other than heads. Moreover, conventional joint estimation approaches for reconstructing all voxels/pixels in PET attenuation and activity maps from only PET emission projection data may result in cross-talk artifacts and incorrect scaling, thus leading to inaccurate attenuation correction, because of under-determinedness and ill-conditionedness of the corresponding inverse problem.

Further, in certain scenarios, MRI may provide only a truncated field of view (FOV) and may not provide information corresponding to extra-patient components such as beds and coils. The truncated part and the extra-patient components may contribute to photon attenuation, which may lead to degraded image quality and inaccurate PET quantitation.

### BRIEF DESCRIPTION

Certain aspects of the present disclosure are drawn to methods, systems and non-transitory computer readable media for imaging are disclosed. To that end, emission projection data corresponding to a target region of a subject is acquired using an emission tomography system. Additionally, one or more magnetic resonance images of the target region are generated using a magnetic resonance imaging system operatively coupled to the emission tomography system. A partially-determined attenuation map is determined by identifying one or more regions in the partially-determined attenuation map with a designated confidence level based on the magnetic resonance images. Further, a complete attenuation map and/or a complete activity map are reconstructed from the emission projection data using the partially-determined attenuation map as a constraint. One or more images corresponding to the target region are then generated based on the partially-determined attenuation map, the complete attenuation map and/or the complete activity map.

### DRAWINGS

These and other features and aspects of embodiments of the present technique will become better understood when the following detailed description is read with reference to the accompanying drawings in which like characters represent like parts throughout the drawings, wherein:

FIG. 1 is a pictorial view of an embodiment of a hybrid PET/MRI system configured to obtain attenuation-corrected PET and PET/MR images, in accordance with an aspect of the present disclosure;

FIG. 2 is a flowchart depicting an exemplary method for enhanced tomographic imaging, in accordance with aspects of the present disclosure;

FIG. 3 is a schematic representation of an embodiment of an undetermined PET attenuation map; and

FIG. 4 is a schematic representation of an embodiment of an MR image;

FIG. 5 is a schematic representation of another embodiment of an MR image with one or more regions identified with a designated confidence level, in accordance with aspects of the present disclosure;

FIG. 6 is a schematic representation of an embodiment of a partially-determined attenuation map based on information derived from the regions identified from one or more MR images, in accordance with aspects of the present disclosure;

FIG. 7 is a schematic representation of embodiments of a true attenuation map and a true activity map;

FIG. 8 is a schematic representation of another embodiment of a partially-determined attenuation map, in accordance with aspects of the present disclosure; and

FIG. 9 is a schematic representation of embodiments of a complete attenuation map and a complete activity map, in accordance with aspects of the present disclosure.

### DETAILED DESCRIPTION

The following description presents exemplary systems and methods for attenuation correction in nuclear images using MR data. Particularly, embodiments illustrated hereinafter disclose hybrid PET/MRI systems and methods that allow for enhanced PET imaging by simultaneously generating an emission activity map and an attenuation map from emission data and a partially-determined attenuation map.

Although exemplary embodiments of the present technique are described in the context of correction of PET and/or PET/MR images, the approaches described herein are also applicable to attenuation correction and/or image modification in other modalities, such as single photon emission computed tomography (SPECT). Therefore, while hybrid PET/MR imaging is presently discussed, it should be noted that the disclosed techniques are also applicable to hybrid SPECT/MR, SPECT image modification and/or attenuation correction, and any other imaging modality in which attenuation correction or attenuation-based modifications may be desirable. Further, in addition to medical imaging, embodiments of the systems and methods discussed herein may be used in pharmacological and pre-clinical research for the development and evaluation of innovative tracer compounds.

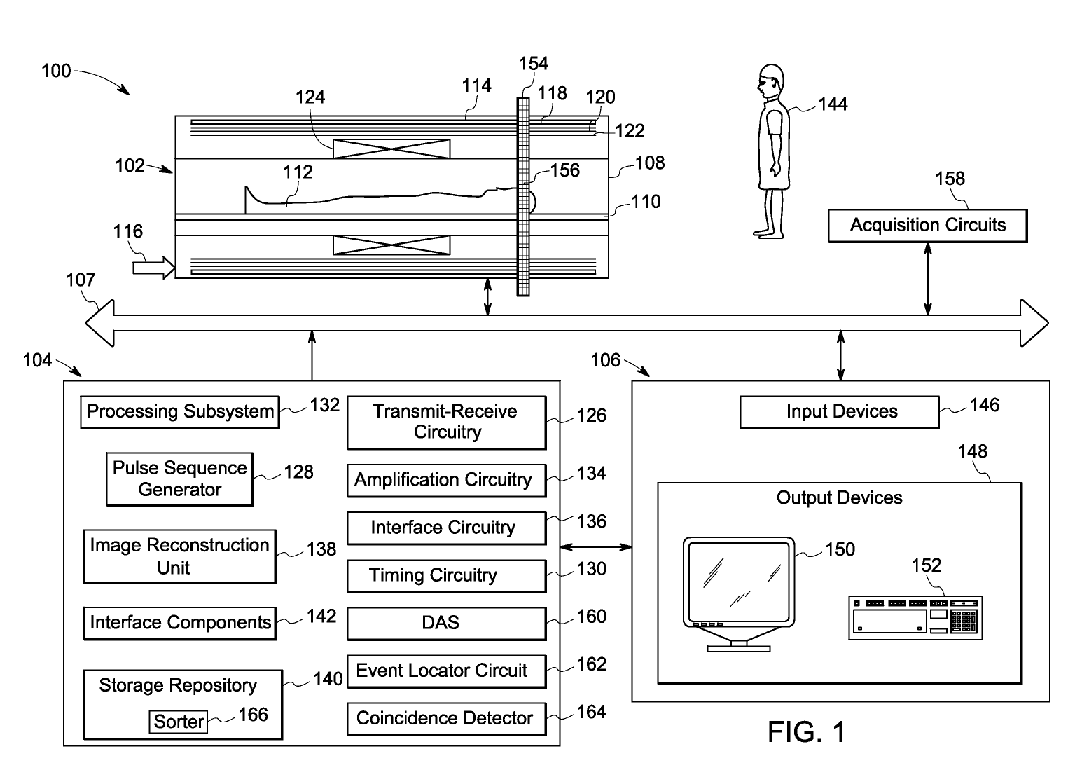

It may also be noted that the embodiments of image data acquisition described herein may be performed sequentially, such as by first obtaining PET image data followed by the acquisition of MR image data, or vice versa. Alternatively, the image data acquisition may be performed substantially simultaneously via simultaneous acquisition of PET and MR image data. The acquisition of both types of image data may enable the generation of images having spatial resolution and structural data associated with MR scans, while also including functional data produced by PET scans. Accordingly, in certain implementations, a PET image produced from a given PET scan may be attenuation-corrected using MR data collected at substantially the same time as the PET scan. An exemplary environment that is suitable for practicing various implementations of the present disclosure is discussed in the following sections with reference to FIG. 1.

FIG. 1 illustrates an exemplary imaging system 100 for enhanced nuclear imaging of a subject of interest. In one embodiment, the system 100 corresponds to a hybrid PET/MR system configured to generate an emission activity map and an attenuation map from emission data and a partially-determined attenuation map. Although the embodiment, illustrated in FIG. 1 illustrates an integrated PET/MR system, in certain embodiments, independent PET and MR systems may be employed for imaging the subject.

To that end, in one embodiment, the hybrid PET/MR imaging system 100 includes a scanner 102, a system controller 104 and an operator interface 106 communicatively coupled to each other over a communications link and/or a communications network 107. In certain embodiments, the system controller 104 may be configured to perform MR and PET imaging, for example, using imaging sequences capable of generating in-phase, out-of-phase, water, fat, and functional PET images. In one embodiment, the PET/MR system 100 may be configured to generate at least the MR images within the same repetition time (TR). Particularly, the PET/MR system 100 may be configured to perform sequences such as Liver Acquisition with Volume Acquisition (LAVA) and LAVA flex sequences, and/or use reconstruction techniques such as Dixon and/or Iterative Decomposition of water and fat with Echo Asymmetry and Least squares estimation (IDEAL) techniques. Additionally, in certain embodiments, the MRI contrast may be T1-weighted (T1w), proton density weighted (PDw), T2 weighted (T2w), and/or may be optimized for segmenting a particular tissue type and/or to avoid certain artifacts.

Although the embodiment illustrated in FIG. 1 depicts a full body scanner 102, in certain embodiments, the MRI system 100 may include any suitable MRI scanner based on specific imaging and/or examination requirements. Further, a presently contemplated configuration of the MRI system 100 is drawn to a horizontal cylindrical bore imaging system employing a superconducting primary field magnet assembly. Certain other embodiments, however, may employ various other system configurations based on specific imaging mandates. The MRI system 100, for example, may include scanners employing vertical fields generated by superconducting magnets, permanent magnets and/or electromagnets.

Additionally, while FIG. 1 illustrates a closed MRI system 100, certain embodiments of the present disclosure may also be used in an open MRI system designed to allow access to a physician, such as during interventional imaging. It may also be noted that in certain embodiments, the MRI system 100 may include any suitable MRI scanner configuration in lieu of the full body scanner 102 illustrated in FIG. 1 based on specific imaging and/or examination requirements.

Further, in certain embodiments, the scanner 102 may include a patient bore 108 into which a table 110 may be positioned for disposing the subject such as a patient 112 in a desired position for scanning. Moreover, the scanner 102 may also include a series of associated coils for imaging the patient 112. Particularly, in one embodiment, the scanner 102 includes a primary magnet coil 114, for example, energized via a power supply 116 for generating a primary magnetic field generally aligned with the patient bore 108. The scanner 102 may further include a series of gradient coils 118, 120 and 122 grouped in a coil assembly for generating accurately controlled magnetic fields, the strength of which vary over a designated field of view (FOV) of the scanner 102.

Particularly, the gradient coils 118, 120 and 122 may have different physical configurations adapted for different functions in the MRI system 100. For example, in one embodiment, the gradient coils 118, 120 and 122 are configured to produce magnetic field gradients used for spatially encoding acquired signals. In certain embodiments, the gradient coils 118, 120 and 122 may have mutually orthogonal axes, allowing a linear field gradient to be imposed in any desired direction using an appropriate combination of the three gradient coils 118, 120 and 122. The field gradient may then be employed for various functions such as slice selection, frequency encoding and/or phase encoding during MR imaging.

Further, the scanner 102 may include an RF coil 124 for generating RF pulses for exciting a gyromagnetic material of interest, typically bound in tissues (gyromagnetic tissue material) of the patient 112. In certain embodiments, the RF coil 124 may also serve as a receiving coil. Accordingly, the RF coil 124 may be operationally coupled to transmit-receive circuitry 126 in passive and active modes for receiving emissions from the gyromagnetic tissue material and for applying RF excitation pulses, respectively. Alternatively, the MRI system 100 may include various configurations of receiving coils different from the RF coil 124. Such receiving coils may include structures specifically adapted for target anatomies, such as head, knee and/or chest coil assemblies. Moreover, receiving coils may be provided in any suitable physical configuration, such as including phased array coils.

In certain embodiments, the system controller 104 controls operation of the associated MR coils for generating desired magnetic field and RF pulses. To that end, in one embodiment, the system controller 104 may include a pulse sequence generator 128, timing circuitry 130 and a processing subsystem 132 for generating and controlling imaging gradient waveforms and RF pulse sequences employed during patient examination. In one embodiment, the system controller 104 may also include amplification circuitry 134 and interface circuitry 136 for controlling and interfacing between the pulse sequence generator 128 and the coils of the scanner 102. The amplification circuitry 134 may include one or more amplifiers that process the imaging gradient waveforms for supplying desired drive current to each of the gradient coils 118, 120 and 122 in response to control signals received from the processing subsystem 132. In certain embodiments, the amplification circuitry 134 may also amplify and couple the generated RF pulses to the RF coil 124 for transmission.

In one embodiment, the RF coil 124 receives response signals emitted by excited nuclei in the tissues of the patient 112. To that end, the RF coil 124 may be tuned to an imaging resonant frequency of the patient nuclei, for example, to about 63.5 MHz for hydrogen in a 1.5 Tesla magnetic field. In such embodiments, where the RF coil 124 serves both to emit the RF excitation pulses and to receive MR response signals, the interface circuitry 136 may also include a switching device (not shown in FIG. 1) for toggling the RF coil 124 between active/transmitting mode and passive/receiving mode. Additionally, the interface circuitry 136 may include additional amplification circuitry for driving the RF coil 124 and for amplifying the response signals for further processing. In certain embodiments, the amplified response signals may be transmitted to the processing subsystem 132 for determining information for use in image reconstruction.

To that end, the processing subsystem 132, for example, may include one or more application-specific processors, graphical processing units (GPUs), digital signal processors (DSPs), microcomputers, microcontrollers, Application Specific Integrated Circuits (ASICs) and/or Field Programmable Gate Arrays (FPGAs). In one embodiment, the processing subsystem 132 may be configured to use a specific imaging protocol for customizing scan sequences and generating data indicative of the timing, strength and shape of the RF and gradient pulses produced. Additionally, the processing subsystem 132 may ascertain the timing and length of a data acquisition window in the imaging pulse sequence using the timing circuitry 130. The processing subsystem 132 may then process the response signals emitted by excited patient nuclei in response to the RF pulses.

By way of example, in one embodiment, the processing subsystem 132 may be configured to demodulate, filter and/or digitize the response signals for determining the image reconstruction information. To that end, the processing subsystem 132 may be configured to apply analytical routines to the processed information for deriving features of interest, such as location of a stenosis and structural and/or functional parameters such as blood flow in the target ROI. The processing subsystem 132 may be configured to transmit this information to an image reconstruction unit 138 to allow reconstruction of desired images of the target ROI. Additionally, the processing subsystem 132 may be configured to receive and process patient data from a plurality of sensors (not shown in FIG. 1), such as electrocardiogram (ECG) signals from electrodes attached to the patient 112 for display and/or storage.

Accordingly, in certain embodiments, the system controller 104 may further include a storage repository 140 for storing the acquired data, reconstructed images and/or information derived therefrom. The storage repository 140 may also store physical and logical axis configuration parameters, examination pulse sequence descriptions and/or programming routines for use during the scanning sequences implemented by the scanner 102. In certain embodiments, the storage repository 140 may further include programming code for implementing one or more algorithms capable of performing PET image reconstruction based on acquired MR data in accordance with an aspect of the present disclosure. To that end, the storage repository 140 may include devices such as a hard disk drive, a floppy disk drive, a compact disk-read/write (CD-R/W) drive, a Digital Versatile Disc (DVD) drive, a flash drive and/or a solid-state storage device.

In one embodiment, the system controller 104 may include interface components 142 for exchanging the stored information such as scanning parameters and image data with the operator interface 106, for example for use during PET and/or MR imaging. Further, in certain embodiments, the operator interface 106 may allow an operator 144 to specify commands and scanning parameters for use during the interventional procedure. To that end, the operator interface 106 may include at least one operator 144 to configure the system controller 104 to control imaging parameters such as table motion, patient and table orientation, and/or shape and timing of the RF pulse sequences.

Moreover, in certain embodiments, the operator interface 106 may also include output devices 148 such as a display 150 including one or more monitors and/or printers 152. The display 150, for example, may be integrated into wearable eyeglasses, or may be ceiling or cart mounted to allow the interventional practitioner 144 to observe the reconstructed images, data derived from the images and other relevant information such as scanning time throughout the procedure. In one embodiment, the display 150 includes an interactive user interface that may allow selection and display of scanning modes, FOV and prior exam data. The interactive user interface on the display 150 may also allow on-the-fly access to patient data such as respiration and heart rate, scanning parameters and selection of an ROI for subsequent imaging.

As previously noted, during a medical examination, MRI allows determination of structural and/or functional information of the target ROI for diagnosis and/or treatment. In certain embodiments, the structural information derived from MRI images may be used for determining attenuation coefficients for use in PET image reconstruction. To that end, the PET data may be acquired sequentially and/or substantially simultaneously with the MR data acquisition. Particularly, in one embodiment, a positron emitter or a radiotracer may be administered to the patient 112 that targets specific ROIs or tissues of the patient's body.

The PET/MRI system 100, in certain embodiments, may include a detector ring assembly 154 disposed about a patient bore 108 configured to detect radiation events corresponding to the target ROI. The detector ring assembly 154 may include multiple detector rings that are spaced along the central axis to form the detector ring assembly 154. The detector rings, in turn, may be formed of detector modules 156 that include, for example, a 6×6 array of individual bismuth germanate (BGO) detector crystals. The detector crystals detect gamma radiation emitted from a patient, and in response, produce photons.

In one embodiment, the array of detector crystals is positioned in front of a plurality of photomultiplier tubes (PMTs). The PMTs produce analog signals when a scintillation event occurs at one of the detector crystals, for example, when a gamma ray emitted from the patient is received by one of the detector crystals. Further, a set of acquisition circuits 158 in the PET/MRI system 100 receive the analog signals and generate corresponding digital signals indicative of the location and the energy associated with the detected radiation event.

Particularly, in one embodiment, the PET/MRI system 100 includes a data acquisition system (DAS) 160 that periodically samples the digital signals produced by the acquisition circuits 158. The DAS 160, in turn, includes event locator circuits 162 that assemble information corresponding to each valid radiation event into an event data packet. The event data packet, for example, includes a set of digital numbers that precisely indicate the time of the radiation event and the position of the detector crystal that detected the event. Further, the event locator circuits 162 communicate the assembled event data packets to a coincidence detector 164 for determining coincidence events. The coincidence detector 164 determines coincidence event pairs if time and location markers in two event data packets are within certain designated thresholds.

In certain embodiments, the PET/MRI system 100 stores the determined coincidence event pairs in the storage repository 140. The storage repository 140, in one embodiment, includes a sorter 166 to sort the coincidence events in a 3D projection plane format, for example, using a look-up table. Particularly, the sorter 166 orders the detected coincidence event data using one or more parameters such as radius or projection angles for storage. In one embodiment, the processing subsystem 132 processes the stored data to determine time-of-flight (TOF) and/or non-TOF information. The TOF information may allow the PET/MRI system 100 to estimate a point of origin of the electron-positron annihilation with greater accuracy, thus improving event localization.

In certain embodiment, the event localization information may be used to further enhance the quality of PET images reconstructed by the image reconstruction unit 138. In one embodiment, the image reconstruction unit 138 may be an independent device communicatively coupled to the PET/MRI system 100. In another embodiment, the image reconstruction unit 138 may be an integral part of the processing subsystem 132. Alternatively, the processing subsystem 132 may perform one or more functions of the image reconstruction unit 138, including generating one or more PET and/or MR images from the acquired data.

Conventional PET imaging entails reconstruction of a PET activity map that defines a spatial distribution of a radiotracer in the patient body based on the emitted 511 keV photons measured by detector modules 156. The emitted photons that travel through different regions of the patient body or extra-patient components such as tissue, lungs, air, beds and/or MR coils, and thus experience different attenuations. Typically, these attenuation values are corrected for accurate PET quantitation in the activity maps by a segmentation-based approach and/or an atlas-based approach. However, as previously noted, such conventional approaches may result in inaccurate attenuation correction in and/or near bones, metal implants and lungs or may require very sophisticated data processing techniques.

Unlike such conventional approaches, the image reconstruction unit 138 may use embodiments of the present disclosure that allows joint estimation of an emission activity map and an attenuation map from emission data and a partially-determined attenuation map. The partially-determined attenuation map, in turn, may be determined based on acquired MR data. Particularly, in one embodiment, the partially-determined attenuation map may be determined by identifying high-confidence regions with known attenuation values such as water or fat tissue from MR images. The high-confidence regions may be identified, for example, using thresholding, segmentation, atlas-based methods, machine learning and/or using unconventional MR sequences such as ultra-short echo time (UTE) and/or zero echo time (ZTE), followed by assigning determined PET attenuation values to each identified region.

The partially-determined attenuation map and the acquired PET projection data may then be used for reconstructing both the complete emission activity map and the undetermined region of the attenuation map simultaneously. The PET activity map and the attenuation maps may then allow reconstruction of high quality TOF and/or non-TOF PET images and/or provide the operator 144 with corresponding diagnostic information. Further, the reconstructed images may be transmitted to one or more of the output devices 148, such as a display, an audio and/or a video device, for example, coupled to the operator interface 106 in real-time or retrospectively. Communicating the image quality and/or diagnostic information allows a medical practitioner to assess a health condition of the patient 112 and whether values computed from the reconstructed image can be trusted, thus leading to a more informed diagnosis.

It may be noted that the specific arrangements depicted in FIG. 1 are exemplary. Further, the PET/MRI system 100 may be configured or customized for additional functionality, different imaging applications and scanning protocols. Accordingly, in certain embodiments, the PET/MRI system may be coupled to multiple displays, printers, workstations, and/or similar devices located either locally or remotely, for example, within an institution or hospital, or in an entirely different location via one or more configurable wired and/or wireless networks such as the Internet, cloud computing and virtual private networks.

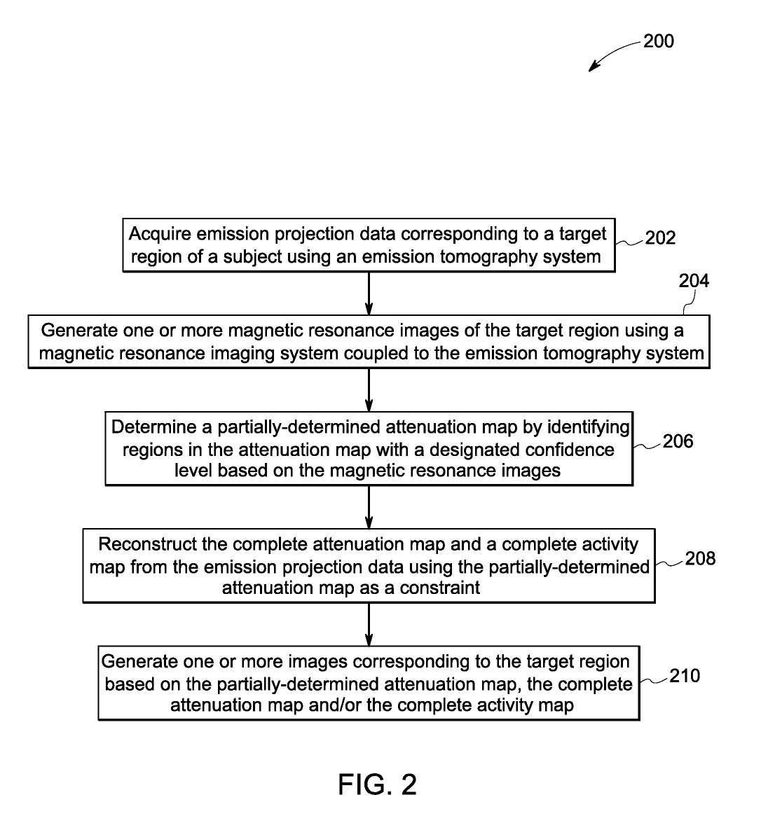

In one embodiment, for example, the PET/MRI system 100 includes, or is coupled to, a picture archiving and communications system (PACS). Particularly, in one exemplary implementation, the PACS is further coupled to a remote system, radiology department information system, hospital information system and/or to an internal or external network to allow operators at different locations to supply commands and parameters and/or gain access to the attenuation corrected PET image data. Certain exemplary methods for improving emission tomographic imaging using joint estimation will be described in greater detail with reference to FIG. 2.

FIG. 2 illustrates a flow chart 200 depicting an exemplary method for improved nuclear imaging. The exemplary method may be described in a general context of computer executable instructions stored and/or executed on a computing system or a processor. Generally, computer executable instructions may include routines, programs, objects, components, data structures, procedures, modules, functions, and the like that perform particular functions or implement particular abstract data types. The exemplary method may also be practiced in a distributed computing environment where optimization functions are performed by remote processing devices that are linked through a wired and/or wireless communication network. In the distributed computing environment, the computer executable instructions may be located in both local and remote computer storage media, including memory storage devices.

Further, in FIG. 2, the exemplary method is illustrated as a collection of blocks in a logical flow chart, which represents operations that may be implemented in hardware, software, or combinations thereof. The various operations are depicted in the blocks to illustrate the functions that are performed, for example, during data acquisition, attenuation and activity map estimation, and image reconstruction phases of the exemplary method. In the context of software, the blocks represent computer instructions that, when executed by one or more processing subsystems, perform the recited operations.

The order in which the exemplary method is described is not intended to be construed as a limitation, and any number of the described blocks may be combined in any order to implement the exemplary method disclosed herein, or an equivalent alternative method. Additionally, certain blocks may be deleted from the exemplary method or augmented by additional blocks with added functionality without departing from the spirit and scope of the subject matter described herein. For discussion purposes, the exemplary method will be described with reference to the elements of FIG. 1.

Generally, tomographic imaging such as PET or SPECT imaging is used to generate two-dimensional (2D) and/or three-dimensional (3D) images for various diagnostic and/or prognostic purposes. Conventional imaging techniques allow for a tradeoff between various imaging criteria such as image quality, spatial resolution, noise, contrast dose and total scanning time. Certain clinical applications, however, entail use of images with high spatial resolution or contrast ratio for investigating minute features within a subject, such as in and around a human heart. Particularly, clinical decisions regarding diagnosis and treatment of detected disease conditions are made based on certain image-derived parameters. Typically, clinical decisions entail use of high quality images that accurately characterize the functional and structural parameters of an ROI for use in diagnosis and treatment. Reconstruction of high quality images involves accurate reconstruction of the PET activity map, which in turn, depends upon accurate attenuation correction of PET data.

Accordingly, embodiments of the present method describe an image reconstruction technique that allows for simultaneous estimation of activity and attenuation maps from projection data acquired from an emission-based tomographic system and certain determined information. For discussion purposes, an exemplary embodiment of the present method will be described with reference to attenuation correction of PET images.

At step 202, projection data corresponding to a target region of a subject is acquired using an emission tomography system such as the PET/MRI system 100 of FIG. 1. To that end, a radiopharmaceutical agent (hereinafter referred to as “radiotracer”), for example Fluorodeoxyglucose (FDG), may be administered to the patient for imaging a target ROI of the patient. The radiotracer, for example, may be selected so as to target the specific ROI for imaging. In certain embodiments, the PET/MRI system 100 may acquire the PET projection data corresponding to the target ROI during an estimated decay period of the radiotracer. Specifically, the projection data may provide an estimate of a target specificity of the radiotracer based on measured values representative of tissue radiotracer uptake distribution as a function of time. The measured distribution, in turn, may be used to assess one or more functional and/or physiological parameters, such as blood flow, in the target ROI for use in clinical diagnosis.

Accordingly, in one embodiment, the projection data may be stored as time-of-flight (TOF) information corresponding to a measured difference in time between arrivals of each pair of gamma photons from each annihilation event for reconstructing 2D and/or 3D TOF images with high signal-to-noise ratio (SNR). In another embodiment, the PET/MRI system 100 stores the acquired projection data as non-TOF information. In certain other embodiments, the acquired data may be stored in list-mode format or the acquired data may be gated according to respiratory and/or cardiac gating. The projection data may then be used to reconstruct an emission map and an attenuation map, which in turn, may be used to reconstruct an emission activity map, which defines a spatial distribution of the radiotracer in the patient body based on measurements corresponding to the emitted 511 keV photons.

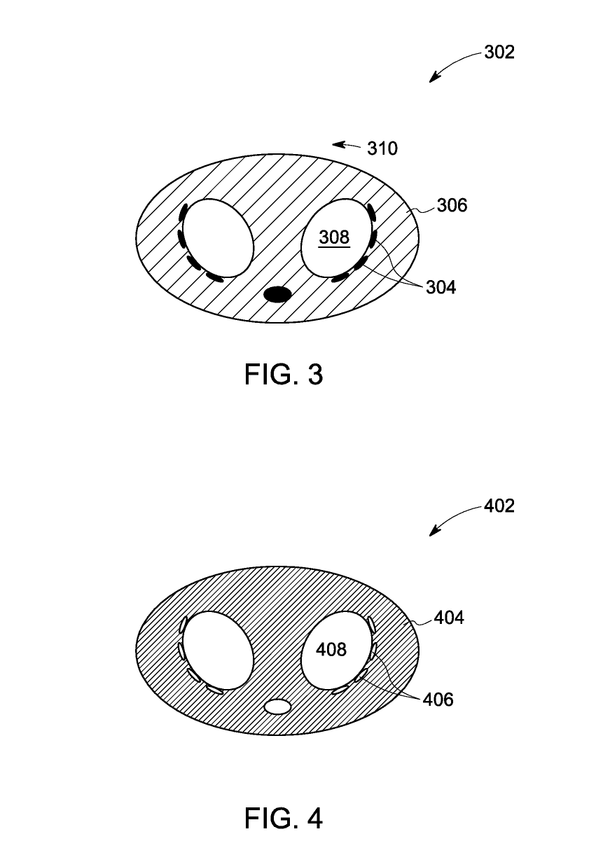

The emitted photons travel through different regions in the patient body and/or extra-patient components such as beds and MR coils, and thus, experience attenuation. The different regions, for example, bones, tissue, lungs and air have different PET attenuation values that may be represented using the PET attenuation map. A schematic representation of an embodiment of an undetermined PET attenuation map 302 illustrated in FIG. 3. As illustrated in the attenuation map 302, typically, bones 304 have a high PET attenuation value, tissue regions 306 have a medium PET attenuation value, lungs 308 have a low PET attenuation value and background air 310 has zero PET attenuation value. For quantitatively accurate PET imaging, these attenuation values are taken into account when reconstructing PET emission activity maps. Particularly, in one embodiment, the PET attenuation map may be reconstructed using MR images and acquired PET projection data.

With returning reference to FIG. 2, at step 204, one or more MR images of the target ROI may be generated using an MRI system, such as the PET/MRI system 100. To that end, the PET/MRI system 100 may scan the target ROI for acquiring MR data before, after or during the PET data acquisition. In one embodiment, the MR data acquisition may entail one or more localization scans, registration scans, and/or pre-processing of MR data, including three-dimensional gradient linearity correction. Further, the MRI scan may acquire one or a combination of in-phase (Iᵢ), out-of-phase (Iₒ), water (I_{w}), and fat (I_{f}) images.

In one embodiment, the MR data acquisition performed within a single TR may obtain all four images for a particular slice selection, and may be extended for generating MR images of the whole body of the patient. The MR images may provide anatomical information with high spatial resolution. MR images, however, have low signal values in bones, lungs and air. Particularly, unlike PET images in which the bones, and air and/or lungs have significantly different PET attenuation values, in MR images bones, lungs and air have similar low signal values, and thus, are difficult to differentiate.

FIG. 4, for example, illustrates a schematic representation of an embodiment of an MR image 402. As illustrated in the MR image 402, soft tissue regions 404 have high MR signal values, whereas bones 406 and lungs 408 have similar low signal values, and thus, it is difficult to differentiate between bones and lungs in the MR image 402. If the MR image 402 could be segmented into fat, water, bones and lungs accurately, a complete PET attenuation map could be generated by assigning determined PET attenuation values to each segment. MR and PET systems, however, employ different imaging principles. Conventionally, transformation of MR images into PET attenuation maps, thus, is not straightforward.

Embodiments of the present disclosure, however, allow for use of the MR images along with the PET projection data in reconstructing accurate PET attenuation maps. To that end, in certain embodiments, the generated MR images may be transformed by image registration or a determined geometrical calibration such that the MR images and the PET attenuation and activity maps are in a common spatial coordinate system, particularly when MR and PET scans are sequentially performed using separate scanning devices.

Returning to FIG. 2, at step 206, a partially-determined attenuation map may be determined by identifying one or more regions in the attenuation map with a designated level of confidence using the MR images. Typically, the MR images provide anatomical information with high spatial resolution and soft tissue contrast. Accordingly, water or fat tissues may be identified from the MR images. In one embodiment, for example, a particular region may be identified as a tissue region if MR signals corresponding to the particular region exceed a designated threshold. The designated threshold, in certain embodiments, may be chosen to be conservative such that the identified tissue region is unlikely to include bones, lungs and air, whereas remaining regions in the MR image with signal values smaller than the designated threshold are likely to be bones, lungs, air and certain tissues. Therefore, in these embodiments, larger the designated threshold, larger is a corresponding confidence level used for reliable determination of the identified regions. As previously noted, in certain embodiments, the LAVA flex sequence or IDEAL MRI technique may be used to partially determine water and fat regions in the PET attenuation map with the desired confidence level.

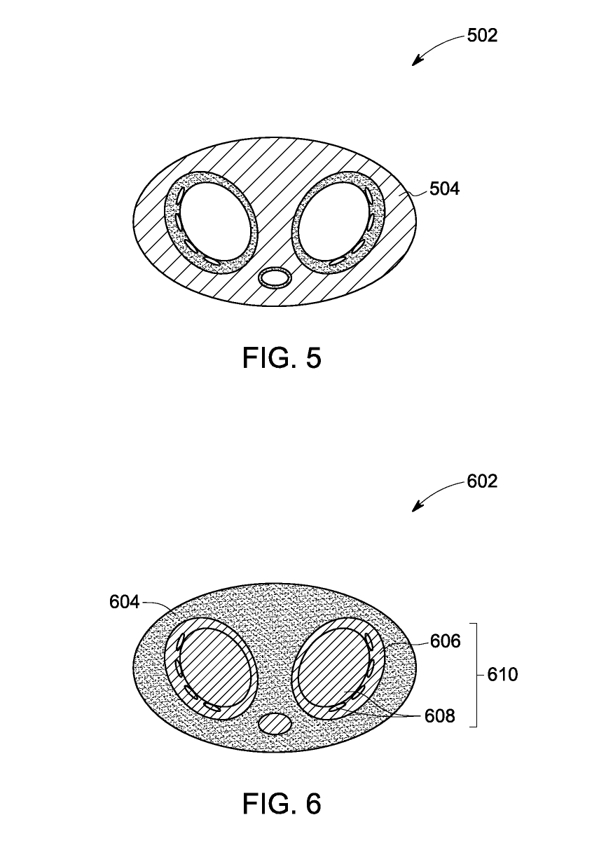

FIG. 5 illustrates a schematic representation of another embodiment of an MR image 502 including a region 504 identified as a tissue with a high confidence level. In certain embodiments, PET/MRI system 100 assigns a pre-determined tissue attenuation value to the region 504 identified as tissue in the MR image 502. Moving to FIG. 6, the PET/MRI system 100 generates a partially-determined attenuation map 602 based on the information derived from the regions identified from the MR image 502 of FIG. 5. The PET/MRI system 100 may determine the complete PET attenuation map, or only a portion thereof.

Particularly, in one embodiment, the PET/MRI system 100 determines only a portion 604 of the attenuation map 602 corresponding to those regions 504 in the MR image 502 of FIG. 5 that were identified with high confidence. For example, in FIG. 6, tissue regions 606 proximal to low MR signal regions 608, representative of bones and lungs, are excluded from the tissue region 604 identified with a high confidence level as there may be some uncertainty or error in boundary regions 606 between the tissue regions 604 and the low MR signal regions 608. Although, a presently contemplated embodiment describes the use of thresholding for identifying the tissue regions 604, in certain embodiments, one or more other regions including tissues may be identified using certain other techniques.

For example, in certain embodiments, regions such as bone, air and lungs may be identified by segmentation methods, atlas-based methods, machine learning methods and/or using unconventional MR sequences such as ultra-short echo time (UTE) and zero echo time (ZTE) sequences. Further, predetermined PET attenuation values may be assigned to each of these identified regions. However, regions 610 that may not be determined with a high confidence level, such as boundary regions 606 between identified regions, may remain undetermined in the partial attenuation map 602.

Returning to FIG. 2, at step 208, the complete attenuation map and a complete activity map are reconstructed from the emission projection data using the partially-determined attenuation map as a constraint. Particularly, in one embodiment, the undetermined regions in the attenuation map and the activity map may be reconstructed from the PET projection data using penalized-likelihood (also known as maximum a posteriori) with a constraint of the partially-determined attenuation map. In penalized-likelihood, an objective function may be chosen. The chosen objective function may include a Poisson log-likelihood function and a regularization function or a penalty function. Further, voxel/pixel values corresponding to an undetermined region of the partially-determined attenuation map and the activity map that maximize the objective function may be determined In one example, the penalized-likelihood objective function with a Gaussian quadratic penalty function may be defined using equation (1),

φ(λ, μ)=L(λ, μ)−β₁R₁(λ)−β₂R₂(μ) (1)

where λ and μ correspond to column vectors representing the emission activity map and the attenuation map, respectively, L corresponds to the Poisson log-likelihood function, R₁ and R₂ correspond to regularization functions for the activity map and the attenuation map, respectively, and β₁ and β₂ correspond to regularization parameters.

For TOF PET projection data, the Poisson log-likelihood function, for example, may be defined using equation (2),

L(λ, μ)=Σ_{i,k}yᵢₖ log(exp(−Σⱼpᵢⱼμⱼ)Σⱼaᵢⱼ⁸λⱼ+rᵢₖ) −(exp(−Σⱼpᵢⱼμⱼ)Σⱼaᵢⱼ^{k}λⱼ+rᵢₖ) (2)

where i and k correspond to sinogram bin index and TOF bin index, respectively, y is representative of the TOF PET projection data, pᵢⱼ corresponds to a geometric forward projection, aᵢⱼ^{k} corresponds to a TOF forward projection including normalization and detector blurring point spread functions, and rᵢₖ corresponds to a mean background contribution of scatter and random coincidences. Although equation (2) illustrates use of TOF PET projection data, in an alternative embodiment, non-TOF PET projection data may be used, and accordingly, the TOF bin index k in equation (2) may be disregarded.

Further, the Gaussian quadratic penalty functions, for example, may be defined using equation (3),

R₁(λ)=Σ_{j,k} wⱼₖ(λⱼ−λₖ)², R₂(μ)=Σ_{j,k} wⱼₖ(μⱼ−μₖ)² (3)

where wⱼₖ corresponds to weights that are non-zero only if voxels j and k are neighbors.

In one embodiment, S may correspond to a set of indices for attenuation map voxels determined at step 206. Particularly, for j S, the attenuation value for voxel j may be determined as μⱼ=μⱼ^{known}. Furthermore, the reconstructed activity map λ^{recon} and the reconstructed attenuation map μ^{recon} may be determined by maximizing the penalized-likelihood objective function φ(λ, μ) with a constraint {μⱼ=μⱼ^{known}: j S} on the attenuation map. Thus, λ^{recon} and μ^{recon} may be determined by solving a constrained optimization problem, for example, defined using equation (4),

maximize φ(λ, μ) subject to λⱼ≧0 and μⱼ≧0 for all j, and μⱼ=μⱼ^{known} for j S (4)

For regularization functions, for example, including non-quadratic non-Gaussian penalty functions having edge-preserving properties such as Huber and log cosh functions, generalized Gaussian and relative difference penalty functions may be used. In certain embodiments, uni-modal or multi-modal distribution functions that represent a probability distribution of the attenuation values may be used for the regularization functions. In certain further embodiments, a combination of aforementioned regularization functions may be used. Alternatively, a zero function may be used for the regularization functions, and in such a scenario, penalized-likelihood estimation reduces to maximum-likelihood estimation. For maximum-likelihood estimations, regularization techniques such as terminating early numerical algorithms, sieves and post-smoothing may be used. In certain other embodiments, weighted least squares may be used instead of the Poisson log-likelihood; in which case, penalized-likelihood estimation becomes penalized weighted least squares estimation of the emission activity map and the undetermined part of the attenuation map.

In a presently contemplated embodiment, the partially-determined attenuation map from 104 may be used as a hard constraint for the optimization problem for reconstructing the emission activity map and the undetermined part of the attenuation map. In an alternative embodiment, however, the partially-determined attenuation map may be used as a soft constraint. Accordingly, a penalty function that penalizes a deviation from the constraint may be added to the penalized-likelihood objective function. In one example, such a penalty function may be defined using equation (5),

R^{soft constraint}(μ)=βΣⱼ s(μⱼ−μⱼ^{known})² (5)

In certain embodiments, alternative dissimilarity measures for the distance between μⱼ and μⱼ^{known} including norms, seminorm, mutual information and cross entropy may be used.

In certain embodiments, the penalized-likelihood estimates of the emission activity map and the undetermined part of the attenuation map may be determined by solving the constrained optimization problem by using a numerical optimization algorithm. In one embodiment, for example, the activity map and the undetermined part of the attenuation map may be updated alternately until one or more designated criteria are satisfied. To that end, the activity map may be updated, for example, by De Pierro's modified expectation maximization (EM) algorithm, whereas the undetermined part of the attenuation map can be updated, for example, by separable paraboloidal surrogates (SPS) algorithm. Additionally, in certain embodiments, only a subset of the acquired PET projection data may be used for each update for accelerating the convergence of the optimization algorithm that allows estimation of the emission activity map and the undetermined part of the attenuation map. In certain other embodiments, the activity map and the attenuation map may be updated using preconditioned conjugate gradient (PCG), ordered subsets expectation and maximization (OSEM), or block sequential regularized expectation maximization (BSREM) algorithm.

Further, at step 210, one or more images corresponding to the target region may be reconstructed using the partially-determined attenuation map, the complete attenuation map and/or the complete activity map. In one embodiment, for example, the emission activity map may be re-reconstructed from the acquired PET projection data using the reconstructed attenuation map and emission tomography image reconstruction algorithms such as OSEM, BSREM, PCG, SPS and its ordered subsets (OS) version and/or De Pierro's modified expectation maximization and its OS version.

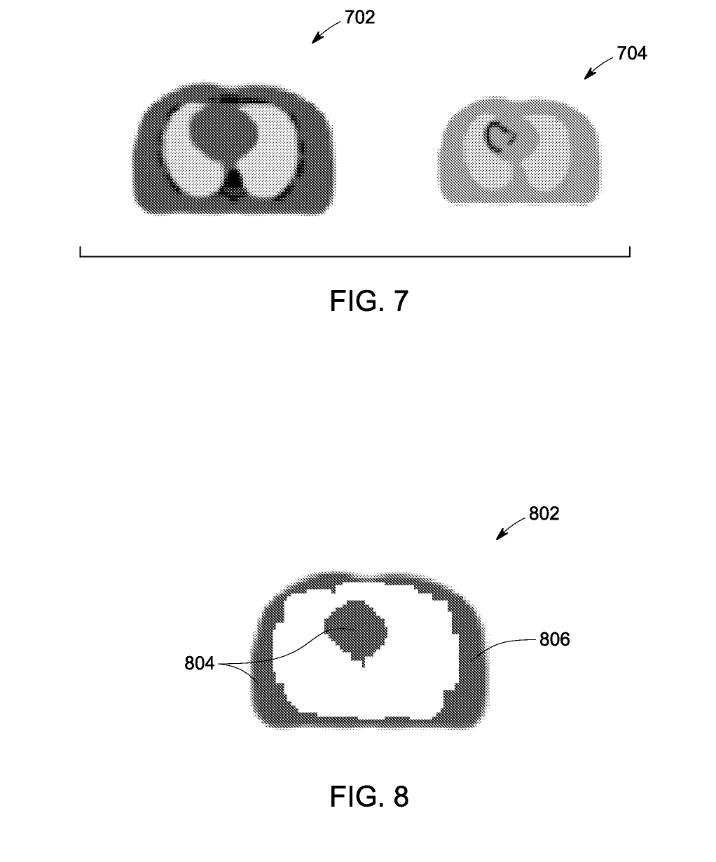

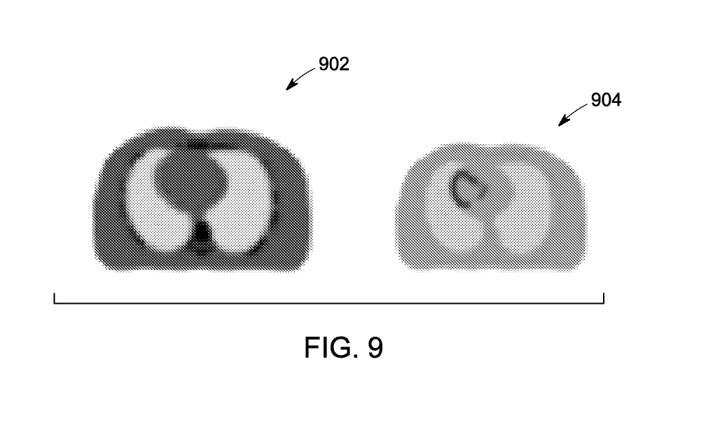

FIG. 7 illustrates a true attenuation map 702 and a true activity map 704, which were used to generate simulated TOF PET projection data. Further, FIG. 8 illustrates a partially-determined attenuation map 802 generated from one exemplary implementation of the present method. In the partially-determined attenuation map 802, as previously noted, a tissue region 804 was identified and a pre-determined tissue attenuation value was assigned to the identified tissue region 804. The undetermined region 806 in the attenuation map 802 included bones, lungs and even tissue. The partially-determined attenuation map 802 was then used to generate a complete attenuation map 902 and a complete activity map 904 illustrated in FIG. 9.

Specifically, the complete attenuation map 902 and the complete activity map 904 were obtained by maximizing a penalized-likelihood objective function with a constraint of the partially-determined attenuation map 802 by alternating between an update of the activity map 904 using De Pierro's modified expectation maximization and an update of the undetermined part of the attenuation map 802 of FIG. 8 using separable paraboloidal surrogates algorithm. Use of embodiments of the present method allowed accurate recovery of bones and lungs as illustrated by the completed attenuation map 902.

Embodiments of the present systems and methods, thus, describe a nuclear imaging technique for simultaneous estimation of the PET emission activity and PET attenuation maps from PET emission projection data and a partially-determined PET attenuation map that is obtained from MR images. Particularly, embodiments described herein allow identification of bones, metal implants, air and lungs in the patient PET attenuation map and corresponding heterogeneous regions, typically indistinguishable in MR images. Additionally, embodiments of the present systems and methods may also allow identification of extra-patient components such as beds and MR coils in the PET attenuation map, which are also difficult to identify in MR images. Although the present description is drawn to PET imaging, embodiments of the present systems and methods may also apply to SPECT imaging where SPECT emission activity and SPECT attenuation maps may be reconstructed using SPECT projection data and MR images.

Although specific features of various embodiments of the present systems and methods may be shown in and/or described with respect to only certain drawings and not in others, this is for convenience only. It is to be understood that the described features, structures, and/or characteristics may be combined and/or used interchangeably in any suitable manner in the various embodiments, for example, to construct additional assemblies and techniques. Furthermore, the foregoing examples, demonstrations, and process steps, for example, those that may be performed by the system controller 104, the processing subsystem 132, the DAS 160 and the image reconstruction unit 138 may be implemented by a single device or a plurality of devices using suitable code on a processor-based system.

It should also be noted that different implementations of the present disclosure may perform some or all of the steps described herein in different orders or substantially concurrently, that is, in parallel. In addition, the functions may be implemented in a variety of programming languages, including but not limited to Python, C++ or Java. Such code may be stored or adapted for storage on one or more tangible, machine-readable media, such as on data repository chips, local or remote hard disks, optical disks (that is, CDs or DVDs), solid-state drives or other media, which may be accessed by a processor-based system to execute the stored code.

While only certain features of the present invention have been illustrated and described herein, many modifications and changes will occur to those skilled in the art. It is, therefore, to be understood that the appended claims are intended to cover all such modifications and changes as fall within the true spirit of the invention.

1. A method, comprising:
acquiring emission projection data corresponding to a target region of a subject using an emission tomography system;
generating one or more magnetic resonance images of the target region using a magnetic resonance imaging system, wherein the emission tomography system is operatively coupled to the magnetic resonance imaging system;
determining a partially-determined attenuation map by identifying one or more regions in the partially-determined attenuation map with a designated confidence level based on the magnetic resonance images;
reconstructing a complete attenuation map, a complete activity map, or a combination thereof, from the emission projection data using the partially-determined attenuation map as a constraint; and
generating one or more images corresponding to the target region based on the partially-determined attenuation map, the complete attenuation map, the complete activity map, or combinations thereof.

2. The method of claim 1, wherein the emission projection data and magnetic resonance imaging data are acquired simultaneously.

3. The method of claim 1, wherein the emission projection data and magnetic resonance imaging data are acquired sequentially.

4. The method of claim 1, wherein acquiring projection data comprises acquiring time-of-flight projection data by scanning one or more views of the subject.

5. The method of claim 1, wherein acquiring projection data comprises acquiring non-time-of-flight projection data by scanning one or more views of the subject.

6. The method of claim 1, wherein the generating one or more images comprises iterative image reconstruction, regularized image reconstruction, model-based image reconstruction, penalized-likelihood image reconstruction, ordered subset expectation maximization-based reconstruction, block sequential regularized expectation maximization-based reconstruction, ordered subset maximum a posteriori expectation maximization-based reconstruction, preconditioned conjugate gradient-based image reconstruction, ordered subset separable paraboloidal surrogate-based image reconstruction, or combinations thereof.

7. The method of claim 1, further comprising administering a radiopharmaceutical to the subject being imaged.

8. The method of claim 1, further comprising associating a determined attenuation coefficient with one or more of the identified regions in the attenuation map.

9. The method of claim 1, further comprising identifying one or more regions in the attenuation map with the designated confidence level using thresholding, segmentation, atlas-based methods, machine-learning, pattern-recognition, ultra-short echo time magnetic resonance sequences, zero echo time sequences, or combinations thereof.

10. The method of claim 1, further comprising registering one or more emission images generated using the emission tomography system and the magnetic resonance images on a common spatial coordinate system.

11. The method of claim 1, wherein reconstructing the complete attenuation map, the complete activity map, or the combination thereof, comprises maximizing an objective function using the partially-determined attenuation map as a constraint.

12. The method of claim 11, wherein the objective function comprises a log-likelihood function and a regularization function.

13. The method of claim 11, wherein the complete attenuation map and the complete activity map are updated alternately using the constrained maximization of the objective function until a designated convergence threshold is achieved.

14. The method of claim 13, wherein the objective function is maximized using block sequential regularized expectation maximization, maximizing techniques, or a combination thereof, wherein the maximizing techniques comprise separable paraboloidal surrogates algorithm, De Pierro's modified expectation maximization algorithm, preconditioned conjugate gradient, preconditioned gradient ascent, coordinate ascent, an ordered subset variation of one or more of the maximizing techniques, or combinations thereof.

15. The method of claim 1, wherein reconstructing the complete attenuation map, the complete activity map, or the combination thereof, comprises a penalized-likelihood estimation, a maximum-likelihood estimation, a penalized weighted least squares estimation, or combinations thereof.

16. An imaging system, comprising:
an emission tomography system configured to acquire emission projection data from one or more views corresponding to a target region in a subject;
a magnetic resonance system operatively coupled to the emission tomography system and configured to generate one or more magnetic resonance images of the target region using a magnetic resonance imaging system;
a processing subsystem operationally coupled to one or more of the magnetic resonance system and the emission tomography system, wherein the processing subsystem is configured to:
determine a partially-determined attenuation map by identifying one or more regions in the partially-determined attenuation map with a designated confidence level based on the magnetic resonance images;
reconstruct a complete attenuation map, a complete activity map, or a combination thereof, from the emission projection data using the partially-determined attenuation map as a constraint; and
generate one or more images corresponding to the target region based on the partially-determined attenuation map, the complete attenuation map, the complete activity map, or combinations thereof.

17. The imaging system of claim 16, wherein the imaging system comprises a single or multiple detector imaging system, a positron emission tomography scanner, a single photon emission computed tomography scanner, a dual head coincidence imaging system, or combinations thereof.

18. A non-transitory computer readable medium that stores instructions executable by one or more processors to perform a method for imaging, comprising:
acquiring emission projection data corresponding to a target region of a subject using an emission tomography system;
generating one or more magnetic resonance images of the target region using a magnetic resonance imaging system, wherein the emission tomography system is operatively coupled to the magnetic resonance imaging system;
determining a partially-determined attenuation map by identifying one or more regions in the partially-determined attenuation map with a designated confidence level based on the magnetic resonance images;
reconstructing a complete attenuation map, a complete activity map, or a combination thereof, from the emission projection data using the partially-determined attenuation map as a constraint; and
generating one or more images corresponding to the target region based on the partially-determined attenuation map, the complete attenuation map, the complete activity map, or combinations thereof.
